(19) United States
(12) Patent Application Publication
(10) Pub. No.: US 2024/0293093 A1
(43) Pub. Date: Sep. 5, 2024

(54) COLLAPSIBLE RADIATION SHIELDING DEVICES AND METHODS

(71) Applicant: Radux Devices, LLC, Omaha, NE (US)

(72) Inventors: Andrew Ubel, Omaha, NE (US); Gregory Gordon, Omaha, NE (US)

(21) Appl. No.: 18/268,094

(22) PCT Filed: Dec. 17, 2021
(86) PCT No.: PCT/US2021/064199

§ 371 Date: Jun. 16, 2023

Related U.S. Application Data
(60) Provisional application No. 63/127,002, filed on Dec. 17, 2020.

(51) Int. Cl. A61B 6/10; A61B 46/10; G21F 3/00

(52) U.S. Cl. CPC A61B 6/107; A61B 46/10; G21F 3/00

(57) ABSTRACT

In general, radiation shielding devices that shield radiation from multiple directions are described. In one embodiment, a radiation shielding device is provided, including a first shielding portion, a second shielding portion, and an attachment member configured to support the first shielding portion in a position relative to the second shielding portion. One or more features of the radiation shielding device provide structural support to at least a portion of the radiation shielding device, and/or maintain the radiation shielding device in the selected configuration.

### CROSS-REFERENCE TO RELATED APPLICATIONS

This application claims the benefit of priority to U.S. Provisional Application Ser. No. 63/127,002, filed Dec. 17, 2020, the entirety of which is incorporated herein by reference.

### TECHNICAL FIELD

This document describes devices, systems, and methods for shielding radiation in a medical environment, such as collapsible radiation shielding devices that include a first shielding portion and a second shielding portion for use in shielding healthcare practitioners from radiation.

### BACKGROUND

Healthcare practitioners often work near a radiation field, such as from a fluoroscope, X-ray machine, or other imaging system, when treating a patient. Procedures and therapies are often designed to reduce patient exposure while allowing healthcare practitioners to effectively treat the patient. However, cumulative radiation exposure of physicians and healthcare practitioners may be significant as they often perform multiple treatments in a typical day, and radiation exposure may be increased when a particular treatment requires the healthcare practitioner's body to be close to a field of radiation. For example, the healthcare practitioner's hands may be exposed to radiation from fluoroscopic imaging equipment when inserting a catheter in a patient's vessel, or when delivering other instruments, medicines, fluids, or other endovascular devices in a patient's vessel. Various techniques have been used to limit radiation exposure, such as physical barriers including radiation shielding and bodywear.

### SUMMARY

Some embodiments described herein include collapsible radiation shielding devices and methods that provide protection for a healthcare practitioner, such as a physician, nurse, technician, etc., during a medical procedure. An example radiation shielding device includes one or more foldable regions that facilitate adjustment of the radiation shielding device into a selected configuration by the healthcare practitioner at a time of use. One or more features of the radiation shielding device provide structural support to at least a portion of the radiation shielding device, and/or maintain the radiation shielding device in the selected configuration. In various optional embodiments, the radiation shielding device includes one or more attachment members (e.g., that attach a first shielding portion to a second shielding portion). The radiation shielding device can be manipulated into a selected configuration, such as by unfolding the radiation shielding device from a first, collapsed configuration to a second, expanded configuration. The attachment members at least partially maintain a first radiation shielding portion in a selected orientation relative to a second radiation shielding portion. In some embodiments, the radiation shielding device can be manipulated into a selected configuration, such as by folding the radiation shielding device from a first, planar configuration to a second, non-planar configuration. In various example embodiments, radiation shielding devices and methods described herein facilitates efficient adjustment into a selected configuration in the operating environment with minimal assembly steps, and one or more folds and/or attachment features maintains the selected configuration during a medical operation.

In various example embodiments, the attachment members include one or more of a flexible strap, rigid rod, support panel, adhesive, hook-and-loop, snap-fit, tongue-and-groove, keyed opening, complementary mechanical interface, ball-and-socket, quick-disconnect couplings, etc. The attachment members can prevent relative movement between first and second shielding portions (e.g., when the radiation shielding device is in an expanded configuration) in at least one direction. In some optional embodiments, one or more folded regions, attachment members, and/or stiffening components, alone or together, impart structural support to the radiation shielding device to maintain the selected configuration, without a malleable frame component.

Some example radiation shielding devices described herein can shield radiation from multiple directions, such as by shielding radiation in a generally horizontal orientation (e.g., along a patient) and in a generally vertical orientation (e.g., spanning the gap between a patient and a radiation device). Shielding in multiple orientations, such as generally vertical and horizontal orientations, can achieve increased protection of the healthcare practitioner, while facilitating efficient, patient-centric workflow in the operating environment.

In some optional embodiments, the radiation shielding device can be arranged such that one or more portions are maintained in a selected orientation relative to one or more other portions. For example, the radiation shielding device can be arranged (e.g., by a healthcare practitioner in the operating environment) such that a first radiation shielding portion is maintained in a selected orientation relative to a second radiation shielding portion. The radiation shielding device includes one or more features that maintain the first and/or second portions in the selected orientation. For example, the radiation shielding device includes one or more features that support the first radiation shielding portion in an upwardly-extending orientation, such that the first radiation shielding portion extends at least partially spanning the gap between a patient and a radiation device. In various optional embodiments, the radiation shielding device includes one or more stiffening components, attachment features, structural folds, etc. that at least partially support the first radiation shielding portion in a selected orientation relative to the second radiation shielding portion and/or patient.

In some optional embodiments, the radiation shielding device is manipulable between first and second configurations, such as a flattened or collapsed, storage configuration, and an expanded, operating configuration that provides shielding in multiple directions, without including a malleable frame that is bent at a hinge point when adjusting the radiation shield between the first and second configurations. In an example embodiment, the first and/or second shielding portions include one or more rigid layers, rods, frame components, etc. A flexible region facilitates manipulation of the radiation shielding device into a configuration that includes first and second shielding portions positioned in different orientations. One or more rigid layers, rods, frame components, etc. support the first shielding portion in the selected orientation (e.g., a substantially vertical orientation) relative to the second shielding portion (e.g., in a substantially horizontal orientation/draped over a patient). Alternatively or additionally, the radiation shielding device includes one or more attachment features that facilitate attachment between the first and second shielding portions. When the radiation shield is in a first (e.g., collapsed) configuration, the attachment features are in a first (e.g., unattached) condition, and when the radiation shield is in the second (e.g., expanded) configuration, the attachment features are in a second (e.g., attached) condition. The attachment features at least partially maintain the radiation shielding device in the expanded configuration.

In some example embodiments, the radiation shielding device includes a radiation shielding drape or fabric. The radiation shielding drape or fabric can be non-shape-stable (e.g., such that the radiation shielding drape or fabric can fold, deform, or bend under the force of gravity). One or more stiffening (e.g., rigid) layers, rods, frame components, etc. are optionally included in at least some locations of the radiation shielding drape. For example, in one embodiment, one or more stiffening layers, rods, frame components, etc. are included in a first shielding portion of the radiation shielding device, and the second shielding portion does not include the one or more stiffening layers, rods, frame components, etc.

In some optional embodiments, the first and second shielding portions differ in one or more characteristics. For example, the first shielding portion is relatively more rigid and/or shape-stable, and the second shielding portion is relatively more flexible and/or non-shape-stable radiation shielding drape. Alternatively or additionally, in some embodiments both portions are similarly flexible and/or shape adjustable. In an example embodiment, the first shielding portion includes a first outer surface integral with a second outer surface of the second shielding portion. For example, the first and second shielding portions are portions of an integral radiation shielding drape or fabric. In some embodiments, one or more components are present only in portions of the radiation shielding device, such that different portions of the radiation shielding device have different characteristics.

The first shielding portion is positionable to extend at least partially in a first direction (e.g., a substantially vertical orientation) and the second shielding portion may extend at least partially in a second direction different from the first direction (e.g., at least partially in a substantially horizontal orientation orthogonal to the first shielding portion) when positioned to shield radiation in the medical environment. The shielding device can protect the healthcare practitioner's hands, arms, and body that may otherwise be exposed to relatively higher levels of radiation. In various example configurations, each of the first and second shielding portions include a radiation shielding material such that unsafe levels on first sides of the first and/or second shielding portions may be reduced to safe levels on second sides of the first and/or second shielding portions. For example, the radiation shielding material may include one or more layers or sheets of lead or other radiation blocking metal or material. The radiation shielding material may be laminated or otherwise positioned between outer fabric, plastic, or metal layers. Alternatively or additionally, the radiation shielding material may include a polymeric material infused with one or more materials that sufficiently block radiation to provide a zone of safe radiation levels, such as barium, tin, aluminum, tungsten, lead, other attenuating metal, etc.

In some optional embodiments, the first and second shielding portions of the radiation shielding device are different portions of a single, unitary shielding device. The portions may optionally differ in one or more characteristics, such as a first shielding portion that includes a relatively rigid and/or shape-stable component, and a second shielding portion that is relatively flexible and/or non-shape-stable.

The radiation shielding device can be partially or entirely supported on a patient in close proximity to a radiation device and/or an imaging location. In some optional embodiments, the radiation shielding device is foldable or expandable to provide a selected coverage area. For example, a portion of the radiation shielding device can be folded, unfolded, unrolled, or otherwise expanded (e.g., to provide a second shielding portion) that covers a relatively large area over the patient.

Particular embodiments described herein provide a radiation shielding device, including a first radiation shielding portion, a second radiation shielding attached to the first radiation shielding portion, and an attachment member. The radiation shielding device is adjustable between a first configuration in which the first radiation shielding device is in a first orientation relative to the second radiation shielding portion and a second configuration in which the first radiation shielding device is in a second orientation relative to the second radiation shielding portion. The attachment member is configured to maintain the first radiation shielding portion in the second orientation relative to the second radiation shielding portion.

In some implementations, the radiation shielding device may optionally include one or more of the following features. The first configuration is a flattened or collapsed configuration and the second configuration is a folded or expanded configuration in which the first shielding portion is disposed at an angle relative to the second shielding portion. The angle is between 75° and 105°, in some embodiments. The first and second shielding portions comprise a radiation shielding drape material. The attachment member comprises a flexible strap. The flexible strap is connected to the first shielding portion proximate a first end of the flexible strap and connected to the second shielding portion proximate a second end of the flexible strap. The attachment member comprises a first panel attached to the first shielding portion. The first panel is attachable to the second shielding portion. The first panel is removably attachable to the second shielding portion by a fastener selected from the group consisting of an adhesive, hook-and-loop, snap-fit, button, toggle, ties, and tongue-and-groove fastener. The first panel is foldable relative to the first and/or second shielding portion such that a major surface of the first panel is parallel to a major surface of the first shielding portion when the radiation shielding device is in the collapsed configuration, and angled relative to the first and/or second shielding portion when the radiation shielding device is in an expanded configuration. The major surface of the first panel is parallel with a major surface of the second shielding portion when the radiation shielding device is in a flattened or collapsed configuration, and perpendicular to the major surface of the second shielding portion when the radiation shielding device is in the expanded configuration. The attachment member comprises a fastener configured to attach a bottom surface of the first shielding portion to the second shielding portion. The first shielding portion comprises a surface having a definable fixed curvature. The first shielding portion comprises a plurality of surfaces in a zig-zag pattern. The first shielding portion is a separate component from the second shielding portion. The radiation shield includes a passage defined through a thickness of the first shielding portion, the passage configured to allow medical tool to pass from a first side of the first shielding portion to a second side of the first shielding portion. The first shielding portion is rigid (in at least one direction so as to be self-supporting). The second shielding portion is non-shape stable. The radiation shielding device includes a plurality of foldable regions. The first configuration is a flattened or collapsed configuration in which the radiation shielding device is flat and the second configuration is an expanded configuration in which the radiation shielding device forms a triangle shape. Vertices of the triangle shape are defined by a first foldable region, a second foldable region, and a perimeter edge of the radiation shielding device. The first and second radiation shielding portions comprise an outer fabric layer and an internal radiation shielding layer. The first and second radiation shielding portions comprise a stiffening layer. The first and second radiation shielding portions are integrally formed as a unitary component.

Particular embodiments described herein provide a radiation shielding device, including a radiation shielding drape comprising a first section, a second section, and a third section, the first section comprising a first stiffening component and the second section comprising a second stiffening component, a first foldable region between the first and second sections, and a second foldable region between the second and third sections. The radiation shielding drape is foldable along the first and second foldable regions to adjust the radiation shielding device between a first configuration and a second configuration.

In some implementations, the radiation shielding device may optionally include one or more of the following features. The radiation shielding drape forms a triangle shape in the second configuration. An attachment member is configured to attach the first section to the third section. The attachment member is configured to attach an edge of the first section to the third section. The attachment member is a magnet, a tab, or a fastener. The third section is supportable on an object while the first and second sections extend upwardly above the third section. The object is a patient. A passage is defined through a thickness of the radiation shielding drape, the passage configured to allow a medical tool to pass from a first side of the radiation shielding drape to a second side of the radiation shielding drape. The first and second shielding inserts are rigid. The third section is flexible.

Particular embodiments described herein provide a method of shielding radiation, including adjusting a first radiation shielding portion of a radiation shielding device relative to a second radiation shielding portion of the radiation shielding device, and positioning an attachment member to extend between the first radiation shielding portion and the second radiation shielding portion.

In some implementations, the method may optionally include one or more of the following features. Positioning the attachment member includes folding or unfolding a panel of the radiation shielding device to at least partially maintain the first radiation shielding portion in a selected position relative to the second radiation shielding portion. The method further includes positioning the radiation shielding device on a patient.

Particular embodiments described herein provide a radiation shielding device, including first and second radiation shielding portions and means for maintaining the first radiation shielding portion in a selected position relative to the second radiation shielding portion. In some embodiments, the radiation shielding device does not include a malleable, shape-stable component at the hinge point between the first and second portions.

Some embodiments of the devices, systems and techniques described herein may provide one or more of the following advantages. First, some embodiments described herein provide a zone of radiation shielding for a healthcare practitioner in the operating environment, while facilitating efficient set-up and ergonomic workflow. An example radiation shielding device can be efficiently manipulated and supported in an expanded configuration that provides radiation shielding in multiple directions (e.g., generally vertical and horizontal directions) and that in some configurations span the gap between the patient and a radiation device.

Second, some embodiments described herein may facilitate positioning of a radiation shielding device in a selected configuration. For example, the radiation shielding device includes one or more foldable regions that facilitate manipulation into a selected configuration in which a first shielding portion is oriented at an angle relative to a second shielding portion. Alternatively or additionally, the foldable regions can provide structural support to the radiation shielding device, such as by providing a three-dimensional radiation shielding device that is self-supporting. Some example embodiments include one or more attachment features that maintain the radiation shielding device in the selected configuration during a medical operation. In some embodiments, the selected configuration can be maintained without requiring manipulation of a malleable frame component (e.g., a bendable metal component).

Third, some example embodiments facilitate compact storage and transport. For example, some example radiation shielding devices having one or more foldable regions facilitate packaging in a flattened or collapsed configuration in which the device is substantially flat. Such a configuration can reduce packaging material and product volume during shipping and storage. At a time of use, the radiation shielding device can be adjusted into an expanded configuration with limited or no assembly required.

Fourth, some example embodiments facilitate efficient manufacturing. For example, some radiation shielding devices can provide a unitary construction that includes first and second shielding portions. For example, a drape or shielding material can be manufactured in bulk and cut to size for each individual radiation shielding device. The foldable regions can be provided intermittently at one or more locations on the radiation shielding device and/or the entire radiation shielding device can be foldable (e.g., at locations between discrete stiffening elements). Such a construction can reduce manufacturing steps and material waste, while also reducing assembly steps for an end user.

Fifth, some example embodiments provide a lightweight and portable radiation shielding device. The radiation shielding device can be partially or entirely supported on a patient, while providing a zone of radiation shielding for the healthcare practitioner that includes shielding in multiple directions (e.g., generally vertical and generally horizontal directions).

Sixth some embodiments described herein provide a high degree of radiation shielding while facilitating efficient operation by the healthcare practitioner. For example, the radiation shielding device may include a substantially continuous or unitary outer layer and/or substantially continuous radiation shielding layers that provide substantially continuous protection, while including first and second portions adjustable relative to one another into a selected configuration (e.g., such that the first portion is oriented generally horizontally on a patient and the second portion is oriented generally vertically extending away from the patient). In some optional embodiments, the radiation shielding device does not include an unshielded break or opening between first and second shielding portions, for example, and thereby provides a substantially continuous zone of protection for the healthcare practitioner. Furthermore, the radiation shielding device can provide concurrent radiation protection to both the healthcare practitioner and the patient by creating vertical and horizontal barriers.

Seventh, some embodiments described herein facilitate efficient operation in the medical environment. For example, an example radiation shielding device optionally includes a passage through the radiation shield that facilitates passage of a medical device (e.g., a tubular medical device, sheath, interventional tool, or other device) from a first side of the radiation shield (e.g., facing the healthcare practitioner) to a second side of the radiation shield (e.g., in the direct field of radiation below a radiation source). The healthcare practitioner may efficiently and effectively manipulate the device while the device has a direct path to a patient access point or other location within the direct field of radiation. The flexibility in positioning and orienting the radiation shielding device allows the healthcare practitioner to position the device relative to the healthcare practitioner's preferred operating position. In some embodiments, the orientation of one or more radiation shielding devices may further enhance efficient operation by the healthcare practitioner, such as by providing a relatively larger area of protection that facilitates free movement by the healthcare practitioner during a medical procedure while the healthcare practitioner remains in an area substantially shielded from radiation exposure.

Eighth, some embodiments described herein facilitate flexible positioning of the radiation shielding device (e.g., on a surface of the patient) such that the healthcare practitioner can operate from a medically advantageous location of the patient. An operator may thus operate from a location selected primarily based on advantages in patient care while being less constrained by ergonomic or radiation dosage requirements, for example.

Ninth, some embodiments described herein provide a sterile and/or recyclable or disposable radiation shielding device. The radiation shielding device can be packaged in a sterile condition and opened in the medical environment at a time of use. Alternatively or additionally, a constructions that facilitate efficient manufacturing and a reduced number of parts can be readily recycled or disposed (e.g., after a single use) in a cost-effective manner.

Tenth, some embodiments described herein provide a radiation shielding device having a coverage area that can be varied by the healthcare practitioner at the medical location. For example, the radiation shielding device may include a foldable or rollable portion such that a coverage area may be selectively increased or decreased by folding/rolling or unfolding/unrolling the first shielding portion and/or the second shielding portion.

The details of one or more implementations are set forth in the accompanying drawings and the description below. Other features and advantages will be apparent from the description and drawings, and from the claims.

### DESCRIPTION OF DRAWINGS

The present description is further provided with reference to the appended Figures, wherein like structure is referred to by like numerals throughout the several views, and wherein:

FIG. 1 is a perspective view of an example radiation shielding device in a medical environment, including a first shielding portion and a second shielding portion.

FIG. 2A is a top view of the example radiation shielding device of FIG. 1.

FIG. 2B is a perspective view of the example radiation shielding device of FIG. 1.

FIG. 3A is a top view of an example radiation shielding device including a first shielding portion having foldable sides.

FIG. 3B is a perspective view of the example radiation shielding device of FIG. 3A.

FIG. 4A is a perspective view of an example radiation shielding device including a first shielding portion having foldable panels.

FIG. 4B is a front view of the example radiation shielding device of FIG. 4A.

FIG. 5A is a top view of an example radiation shielding device including a curved first shielding portion.

FIG. 5B is a perspective view of the example radiation shielding device of FIG. 5A.

FIG. 6A is a top view of another example radiation shielding device including a first shielding portion and a second shielding portion in a first configuration.

FIG. 6B is a perspective view of the example radiation shielding device of FIG. 6A in a second configuration.

FIG. 7A is a top view of an example radiation shielding device including a first shielding portion having foldable side panels in a first configuration.

FIG. 7B is a perspective view of the example radiation shielding device of FIG. 7A in a second configuration.

FIG. 8A is a top view of another example radiation shielding device including a first shielding portion and a second shielding portion in a first configuration.

FIG. 8B is a perspective view of the example radiation shielding device of FIG. 8A in a second configuration.

### DETAILED DESCRIPTION OF ILLUSTRATIVE EMBODIMENTS

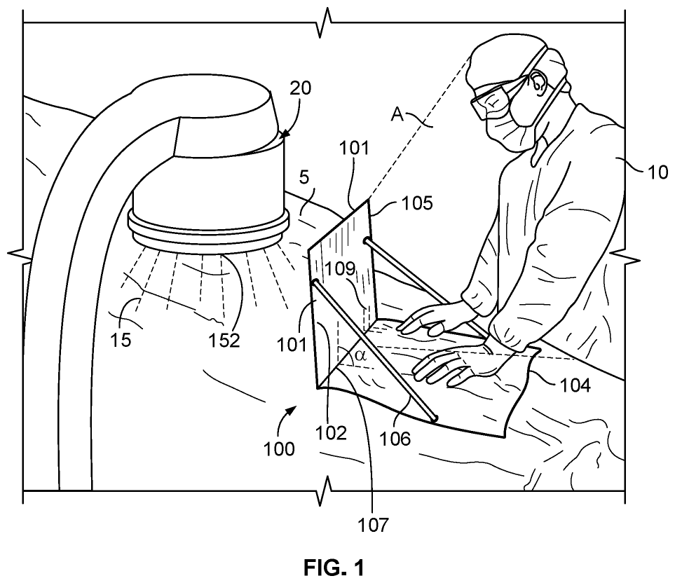

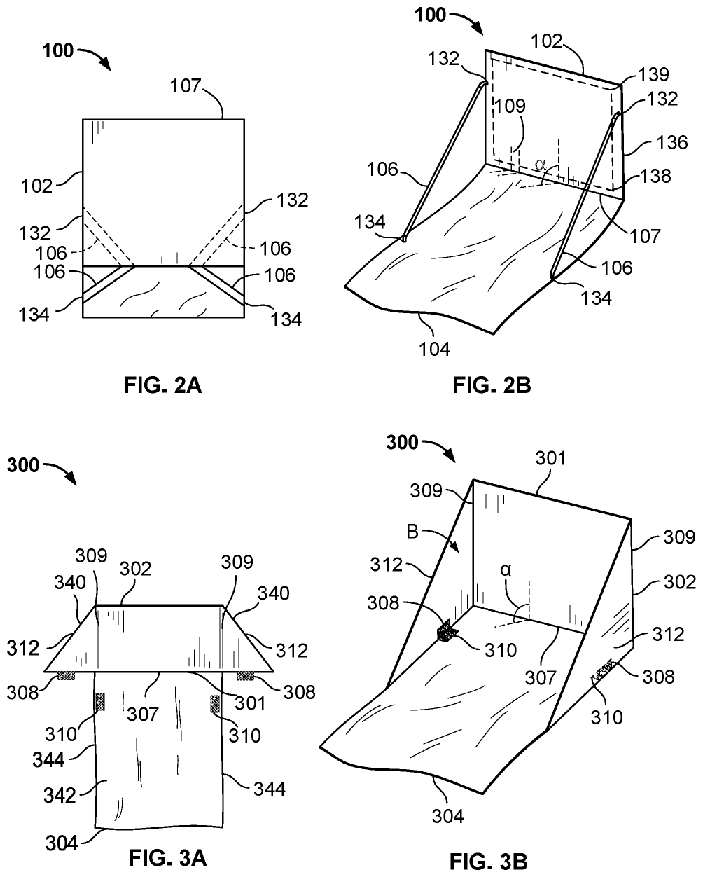

Referring to FIGS. 1, 2A, and 2B, an example radiation shielding device 100 is shown. The radiation shielding device 100 shields the healthcare practitioner 10 from radiation 15 emitted by radiation source 20 when positioned at least partially between a radiation device 20 (e.g., such as a medical imaging device) and a healthcare practitioner 10 during a medical operation. Radiation source 20 can include an emitter configured to emit radiation 15 and/or a detector configured to receive radiation 15 emitted by the emitter. Radiation shielding device 100 provides a radiation shielding zone A in which radiation exposure from radiation device 20 is eliminated or substantially reduced for the healthcare practitioner 10 operating within zone A.

Radiation shielding device 100 is supportable at least partially by a patient undergoing a medical operation. For example, the radiation shielding device 100 is sufficiently compact and lightweight such that the device 100 can be entirely supported on patient 5 (e.g., without an additional base supported by the floor, ceiling, imaging device, or other structure in the medical environment). Such a configuration facilitates efficient workflow by the healthcare practitioner 10 and facilitates positioning of radiation shielding device 100 proximate both radiation source 20 and a target location (e.g., a location being imaged and/or the target of radiation emitted by radiation source 20). Positioning of the radiation shielding device 100 close to the radiation source 20 and/or the target location can provide a relatively large radiation protection zone A while first and second shielding portions 102, 104 may be relatively small.

Radiation shielding device 100 includes a top edge 101 (e.g., a top edge of first shielding portion 102), side edges 103, 105, and a foldable region 107. In use, a healthcare practitioner can select a configuration in which top edge 101 is aligned substantially parallel (e.g., parallel or otherwise within 10° of exactly parallel) with an edge 152 of the radiation source 20 when in the selected orientation. Alternatively, top edge 101 can be angled relative to edge 152 of radiation source 20, such as to align with an angled surface of patient 5 or an object, for example.

Radiation shielding device 100 is positionable substantially between radiation source 20 and healthcare practitioner 10 to shield healthcare practitioner 10 from radiation 15. First shielding portion 102 may extend between patient 5 and radiation source 20 (e.g., extending along more than 75%, more than 85%, more than 95%, about 100%, or more than 100% of a distance between the patient and a height of radiation source 20). For example, healthcare practitioner 10 can adjust radiation source 20 to a height lower than a height of top edge 101 of first shielding portion 102 (e.g., such that edge 152 of radiation source 20 is located at a height between patient 5 and top edge 101 of first shielding portion 102). Alternatively or additionally, a height of top edge 101 can be selected to extend to a height of radiation source 20. First shielding portion 102 arranged to extend between patient 5 and radiation source 20 blocks radiation emitted from radiation source 20 beyond the target area and/or radiation reflected towards the doctor by the patient, operating table, floor, etc. Alternatively, or additionally, second shielding portion 104 can extend from the target area and/or first shielding portion 102 (e.g., proximate foldable region 107 of first shielding portion 102) towards healthcare practitioner 10. The second shielding portion 104 thus blocks radiation reflected towards the healthcare practitioner 10 by patient 5, operating table, floor, etc.

In some embodiments, relative dimensions of first and second shielding portions 102, 104 are selectable by a user by folding the first shielding portion 102 at one of several folding locations. For example, foldable region 107 may include a foldable area that can be folded at a selected location to provide a selected height of first shielding portion 102 (e.g., when the radiation shielding device is in the expanded configuration). In some embodiments, foldable region 107 includes a flexible and/or nor-shape stable region of the radiation shielding device 100. Alternatively or additionally, foldable region may include a region of reduced material thickness, a welded region, or a hinged area.

First and second shielding portions 102, 104 are positionable relative to one another such that radiation shielding device 100 provides a relatively large radiation shielding zone A while being relatively small, lightweight, and/or supportable on patient 5. In an example embodiment, radiation shielding device 100 is adjustable into an expanded configuration in which a first shielding portion 102 is maintained in a selected orientation relative to a second shielding portion 104 by an attachment member 106. For example, radiation shielding device 100 defines an angle (α) between first shielding portion 102 and second shielding portion 104 when in the expanded configuration. In various example embodiments, first shielding portion 102 may be arranged to extended generally upwards to at least partially span a distance between patient 5 and radiation device 20 such that angle (α) is between 45° and 135°, 70° and 110°, 80° and 100°, or about 90°. First shielding portion 102 may be oriented substantially vertically (e.g., vertically or otherwise within 15° of the direction of the gravitational force) and second shielding portion 104 may be oriented substantially horizontally (e.g., horizontally or otherwise within 15° of a direction that is perpendicular to the gravitational force). In some embodiments, the angle (α) is an overall relative orientation between the first and second shielding portions 102, 104, while the first and/or second shielding portions 102, 104 may have non-planar portions or discrete surfaces of varying relative orientations.

In an example embodiment, attachment member 106 comprises one or more flexible straps. Each strap 106 is connected to first shielding portion 102 proximate a first end of the strap and connected to the second shielding portion proximate a second end of the strap. Tension of strap 106 limits relative movement of first shielding portion 102 away from relative to second shielding portion 104 beyond a predetermined point. For example, attachment member 106 limits angle (α) between first and second shielding portions to less than 135° less than 110°, less than 100°, or about 90°. The weight of first shielding portion 102, for example, can cause a tension force in attachment member 106 that maintains radiation shielding device 100 in the expanded configuration during a medical operation. Alternatively or additionally, hinge region 107 may be biased to open first shielding portion 102 away from second shielding portion 104 (e.g., via a spring hinge, leaf hinge, biased stiffener, etc.), and the bias of hinge region 107 causes a tension force in attachment member 106 that maintains radiation shielding device 100 in the expanded configuration. In an example embodiment, the attachment member 106 (e.g., flexible straps) readily flex and bend, and/or do not provide support in compression, such that the first shielding portion 102 can be folded onto the second shielded portion 104 in a collapsed configuration.

In various example embodiments, a length of attachment member 106 between first and second shielding portions 102, 104 is adjustable. For example, first and/or second ends of attachment member 106 are removably attached to first and second shielding portions 102, 104, respectively. The length of attachment member 106 between first and second shielding portions 102, 104 can be increased to increase the angle (α) between first and second shielding portions 102, 104, and decreased to decrease the angle (a) between first and second shielding portions 102, 104.

Alternatively or additionally, attachment member 106 can include a rod, such as an adjustable or telescopic rod (e.g., a rod having an adjustable length). In an example embodiment, the radiation shielding device 100 is adjustable between configurations while first and second ends of the attachment member 106 are attached to the first and second shielding portions 102, 104. The attachment member 106 is expandable in length (e.g., when first shielding device 102 is rotated away from second shielding device 104), and maintain its length against compression, such as by a ratchet mechanism that permits expansion and limits compression. In this way, the healthcare practitioner can expand the radiation shielding device 100 into a selected configuration, and the attachment member supports the first shielding portion in the selected orientation relative to the second shielding portion 104.

In some embodiments, attachment member 106 can include a non-adjustable rod (e.g., a rod having a fixed length), and/or that provides support in both tension and compression. In operation, the radiation shielding device 100 is manipulated into an expanded configuration by the healthcare practitioner. The attachment member 106 may then be attached to the first and/or second shielding portions 102, 104, to maintain the radiation shielding device 100 in the selected configuration, such as be inserting ends of the attachment member 106 into complementary pockets or receptacles of the first and/or second shielding portions 102, 104. In various embodiments, first and second ends of the attachment member can be attached to the first and/or second shielding portions 102, 104 via adhesive, hook-and-loop, snap-fit, button, toggle, ties, keyed opening, complementary mechanical interface, ball-and-socket, quick-disconnect couplings, etc.

Alternatively or additionally, attachment member 106 can include a triangular piece of a fabric that maintains the radiation shielding device 100 in the selected configuration. For example, tension of the fabric limits relative movement of first shielding portion 102 away from relative to second shielding portion 104 beyond a predetermined point.

Attachment member 106 can facilitate efficient set-up and use of radiation shielding device 100. The radiation shielding device 100 allows first and second portions to be oriented relative to one another by the healthcare practitioner, and supported in the selected orientation with little or no assembly. Alternatively or additionally, such constriction can facilitate efficient manufacturing by reducing the number of parts and assembly steps.

During a medical operation, multiple radiation shielding devices 100 can be arranged in close proximity to one another to provide a desired radiation shielding zone A. For example, two radiation shielding devices 100 are positionable at least partially around a perimeter of radiation source 20 (e.g., in a side-by-side arrangement), supported at least partially on a patient. First and/or second portions 102, 104 can be arranged to at least partially overlap to reduce transmission of radiation across the radiation shielding devices 100. The lightweight, portable, and/or patient-supportable configuration of radiation shielding device 100 facilitates flexibility and simplicity in arranging radiation shielding device 100 in the operating environment to provide a customized and relatively large radiation shielding zone A. Radiation shielding device 100 thus can be manipulated into a selected configuration to facilitate a particular medical operation, patient anatomy, operating room workflow, or ergonomic preferences, etc.

In some embodiments, radiation shielding device 100 includes one or more layers of radiation shielding material, such as a sheet of lead or other radiation blocking metal or material. The radiation shielding material may be laminated or otherwise positioned between outer fabric, plastic, non-metallic, or metallic layers. In various example embodiments, radiation shielding device 100 may include one or more of barium, tin, aluminum, tungsten, lead, other attenuating metal, etc. Alternatively, or additionally, radiation shield 106 may include a polymeric material or a fabric material infused or interwoven with one or more materials that sufficiently block radiation to provide a zone of safe radiation levels, such as barium, tin, aluminum, tungsten, lead, or other attenuating material.

Radiation shielding device 100 is configured to facilitate effective imaging of a target area of the patient while providing radiation protection to a healthcare practitioner and/or the patient. In some examples, the radiation shielding device can provide a level of attenuation of at least 50%, 60%, 75%, 80%, or 95% at a radiation level of about 90 kVp (peak kilovoltage), such as directed according to American Society for Testing and Materials (ASTM) Test Method F3094-14. For example, the radiation shielding device can provide a level of attenuation ranging between 50% and 75%, between 75% and 90%, between 90% and 95%, or between 50% to 99% at a radiation level of about 90 kVp. In some examples, the radiation shielding device can provide a level of attenuation of at least 50%, 60%, 75%, 80%, or 95% at a radiation level of about 70 kVp (peak kilovoltage). For example, the radiation shielding device can provide a level of attenuation ranging between 50% and 75%, between 75% and 90%, between 90% and 95%, or between 50% to 99% at a radiation level of about 70 kVp. In some examples, the radiation shielding device can provide a level of attenuation of at least 50%, 60%, 75%, 80%, or 95% at a radiation level of about 105 kVp (peak kilovoltage). For example, the radiation shielding device can provide a level of attenuation ranging between 50% and 75%, between 75% and 90%, between 90% and 95%, or between 50% to 99% at a radiation level of about 70 kVp.

Radiation shielding device 100 includes one or more features that facilitate manipulation of a medical device, such as an interventional tool, by the healthcare practitioner. For example, radiation shielding device 100 includes a passage 109 defined through a thickness of the radiation shielding device to allow passage of the medical device from a first side of radiation shielding device 100 to a second side of radiation shielding device 100. In an example embodiment, passage 109 is located proximate foldable region 107, and extends at least partially through first and second shielding portions 102, 104. Alternatively or additionally, passage 109 is located entirely through first shielding portion 102 and does not extend through second shielding portion 104, or is located entirely through second shielding portion 104 and does not extend through first shielding portion 102. The material of radiation shielding device surrounding passage 109 can be pressed into engagement with the medical device so that little or no gap is present between the medical device and radiation shielding device. In some embodiments, an adhesive or rubberized surface at least partially surrounds passage 109 to facilitate contact between the medical device and radiation shielding device 100.

In an example embodiment, passage 109 is defined by one or more slits through the thickness of radiation shielding device. A portion of radiation shielding device 100 may be folded and/or popped outwardly so that the medical device can pass through radiation shielding device 100. In some embodiments, passage 109 is a slot or a notch that provides a space for a medical device (e.g., a catheter) to pass between first and second shielding portions 102, 104 while simultaneously blocking radiation 15. For example, passage 109 may be shaped and sized slightly larger than a medical device such that the medical device may pass through passage 109. Alternatively, passage 109 may be shaped and sized slightly smaller than the medical device such that medical device is at least partially constrained within passage 109. In some embodiments the passage may be shielded by a flap of radiation shielding material that covers gaps that would otherwise exist around the passage.

In an example embodiment, radiation shielding device 100 includes one or more stiffening layers that impart structure to some or all of radiation shielding device 100. For example, first shielding portion 102 includes a stiffening layer 139. Stiffening layer 139 can include a substantially rigid layer or component, such as a plastic, metal, composite, paperboard, or other stiffening layer. In some embodiments, the stiffening layer 139 is radio-opaque, and can be provided by a radiation shielding layer. In some embodiments, stiffening layer 139 is not malleable (e.g., and returns substantially to its original shape after being bent).

In an example embodiment, stiffening layer 139 is present throughout first shielding portion 102. For example, stiffening layer 139 has dimensions the same or similar as outer dimensions of first shielding portion 102. In some embodiments, stiffening layer 139 includes one or more discrete sheets, rods, frame components, etc., that are spaced apart in first shielding portion 102. The one or more discrete sheets, rods, frame components, etc. can facilitate flexibility radiation shielding device 100 in a first direction, and/or provide support or resist bending in a second direction.

Referring now to FIGS. 2A and 2B, radiation shielding device 100 is adjustable between a first configuration and a second configuration, such as a collapsed configuration (FIG. 2A) and an expanded configuration (FIG. 2B). In an example embodiment, when in the first, collapsed configuration, radiation shielding device 100 is folded (e.g., about a folding axis defined through foldable region 107). The first shielding portion 102 at least partially overlaps with second shielding portion 104, and/or major surfaces of the first and second shielding portions are parallel to one another. In some embodiments, first shielding portion 102 may be folded to extend away from second shielding portion 104 (e.g., when attachment members 106 are not connecting first and second shielding portions 102, 104). For example, first and second shielding portions 102, 104 are co-planar in such an arrangement. Alternatively or additionally, the first configuration is a flat configuration in which radiation shielding can be in a folded configuration or an unfolded configuration.

In some embodiments, the radiation shielding device 100 is entirely or mostly flat when in the first, collapsed configuration. Such a configuration facilitates compact packaging, such as for shipping to a location of use, storage, etc. For example, one or more radiation shielding devices 100 can be packaged in a sterile state at a manufacturing location, and efficiently transported (e.g., in bulk) to locations of use.

Referring now to FIG. 2B, example radiation shielding device 100 is shown in a second configuration. When in the expanded or operating configuration, the first shielding portion 102 is extend upwardly with respect to the second shielding portion 104. Angle (a) defined between first and second shielding portions 102, 104 is between 45° and 135°, 70° and 110°, 80° and 100°, or about 90°, for example. In some embodiments, angle (α) may be selected by healthcare practitioner 10 by adjusting the length of attachment members 106 between first and second shielding portions 102, 104, such as by adjusting the attachment members 106 and/or attachment locations of the attachment members 106 to the first and/or second shielding portions 102, 104. Alternatively, angle (α) may be predetermined by attachment members 106 that have a predefined length and/or are attached (e.g., permanently) at predetermined attachment points 132, 134.

Attachment members 106 are connected to first shielding portion 102 at attachment locations 132 and to second shielding portion 104 at attachment locations 134. In some embodiments, attachment locations 132 and 134 comprise one or more of adhesive, hook-and-loop, snap-fit, button, toggle, ties, keyed opening, complementary mechanical interface, ball-and-socket, quick-disconnect couplings, etc. When in the collapsed configuration (FIG. 2A), attachment members 106 may be folded inwards such that attachment members are sandwiched between first and second shielding portions 102, 104. Alternatively or additionally, attachment members 106 may be folded outwards (e.g., not positioned between the first shielding portion 102 and the second shielding portion 104). In some embodiments, attachment members 106 are removably connectable at one or more of attachment locations 132, 134. For example, radiation shielding device 100 may be packaged in the first configuration (e.g., collapsed configuration) in which attachment members 106 are disconnected. A healthcare practitioner can adjust the radiation shielding device 100 into the second, expanded configuration by connecting attachment members 106 to attachment locations 132, 134, for example.

In an example embodiment, first and/or second shielding portions 102, 104 are expandable or foldable to adjust a coverage area of radiation shielding device 100. For example, second shielding portion 104 may be a flexible shielding fabric (e.g., a drape, an apron, a blanket, a flexible patient radiation shield, etc.) that can be unfolded or expanded to the rear of first shielding portion 102 (e.g., away from second side 138) or to the sides of first shielding portion 102. In some embodiments, the user (e.g., healthcare practitioner 10) may receive example radiation shielding device 100 in a first configuration (e.g., a collapsed configuration) having second shielding portion 104 in a folded or rolled configuration. Healthcare practitioner 10 may subsequently unfold or unroll second shielding portion 104 over a surface of a patient in preparation for a medical operation such that the unfolded or unrolled second shielding portion 104 covers an area of the patient that is relatively larger than an area of the patient that was initially covered by the second shielding portion 104 in the folded or rolled configuration.

In an example embodiment, first and second shielding portions 102, 104 are integrally formed as a unitary component. The first and second shielding portions 102, 104 include one or more layers that extend continuously between the first and second shielding portions 102, 104, such as an outer cover layer, radiation shielding layer, etc. The first and second shielding portions 102, 104 are substantially uninterrupted by separations, gaps, openings, etc., across at least a portion of a width or along folded region 107 of radiation shielding device 100. Example radiation shielding device 100 thus promotes a consistent radiation protection zone to shield healthcare practitioner 10 from radiation 15 emitted by radiation source 20. Alternatively or additionally, first and second shielding portions 102, 104 formed as a unitary component can limit positioning of a portion of radiation shielding device 100 beyond first shielding portion 102 into the target area of radiation source 20. For example, the second shielding portion 104 does not extend beyond first shielding portion 102 on a first side of first shielding portion 102 into the target area of radiation source 20, and extends away from first shielding portion 102 on a second side of first shielding portion 102 opposite the first side.

Referring now to FIGS. 3A and 3B, an example radiation shielding device 300 is shown. Radiation shielding device 300 includes a first shielding portion 302, a second shielding portion 304, and one or more attachment members, such as panels 312, that maintain first shielding portion 302 in a selected orientation relative to second shielding portion 304. In various example embodiments, radiation shielding device 300 includes one or more features as described herein with reference to radiation shielding device 100 described above with reference to FIGS. 1-2.

Radiation shielding device 300 is adjustable between a first configuration and a second configuration, such as a collapsed configuration (FIG. 3A) and an expanded configuration (FIG. 3B). In an example embodiment, when in the first, collapsed configuration, first shielding portion 302 extends away from second shielding portion 304. For example, first and second shielding portions 302, 304, and/or panels 312, are co-planar with one another. Alternatively, radiation shielding device 300 is foldable (e.g., about a folding axis defined through foldable region 307) such that the first shielding portion 302 at least partially overlaps with second shielding portion 304, and/or major surfaces of the first and second shielding portions 302, 304, and/or panels 312, are parallel to one another. In an example embodiment, first and second radiation shielding portions have a generally rectangular outer perimeter shape, and panels 312 have a triangular shape.

In some embodiments, the radiation shielding device 300 is entirely or mostly flat when in the first, collapsed configuration. Such a configuration facilitates compact packaging, such as for shipping to a location of use, storage, etc. For example, one or more radiation shielding devices 300 can be packaged in a sterile state at a manufacturing location, and efficiently transported (e.g., in bulk) to locations of use.

In an example embodiment, radiation shielding device 300 is foldable into a three-dimensional shape that at least partially supports the radiation shielding device 300 in the expanded configuration. Radiation shielding device 300 includes one or more panels 312 that are foldable relative to other portions of radiation shielding device 300. For example, panels 312 are foldable about folding axes located at foldable regions 309.

Referring now to FIG. 3B, example radiation shielding device 300 is shown in a second configuration. When in the expanded or operating configuration, the first shielding portion 302 extends upwardly with respect to the second shielding portion 304. Angle (α) defined between first and second shielding portions 302, 304 is between 45° and 135°, 70° and 110°, 80° and 100°, or about 90°, for example.

The one or more attachment features of radiation shielding device 300 maintains the radiation shielding device in a selected configuration. In an example embodiment, panels 312 are foldable to extend in a non-coplanar direction relative to first and/or second shielding portions 302, 304, to at least partially support first shielding portion 302 in a position relative to second shielding portion 304. For example, panels 312 are foldable to extend between first and second shielding portions 302, 304, such that major surfaces of panels 312 are substantially perpendicular to major surfaces of first and/or second radiation shielding portions 302, 304. Alternatively or additionally, panels 312 are foldable to extend between first shielding portion to an object that supports radiation shielding device 300, such as a patient (e.g., patient draping). The panels 312 can extend outwardly from first shielding portion 302 at an angle greater than 90, such as an angle between 90° and 160°, 110° and 140°, or about 120°. The panels 312 act as a truss or support that limits movement of first shielding portion 302 towards the collapsed configuration (e.g., movement of first shielding portion 302 towards second shielding portion 304 such that angle (α) is reduced).

The attachment features of radiation shielding device 300 include one or more fasteners that can maintain radiation shielding device 300 in the selected configuration. For example, radiation shielding device includes fasteners 310 configured to attached with fasteners 308. In various example embodiments, fasteners 310 include one or more of an adhesive, hook-and-loop, snap-fit, button, toggle, ties, tongue-and-groove, keyed opening, complementary mechanical interface, ball-and-socket, quick-disconnect couplings, etc. In an example embodiment, fastener 310 includes an adhesive strip covered by a release liner. At a time of use, the healthcare practitioner can remove the release liner and bring the adhesive strip into contact with complementary surface of fastener 308 (e.g., an adhesive landing area) to attach panel 312 in a fixed location relative to second shielding portion 304. In an example embodiment, fasteners 308 are positioned along an edge 340 of panels 312, and fasteners 310 are positioned on surface 342 of second shielding portion 304.

Radiation shielding device 300 is constructed of one or more radio-opaque materials, including one or more materials described above with reference to FIGS. 1-2B, to provide radiation shielding for a healthcare practitioner. In an example embodiment, first and second shielding portions 302, 304, including panels 312, include radiation shielding materials (e.g., each have the same radiation-shielding material construction). Alternatively, first and second shielding portions 302, 304 include one or more radiation shielding materials that are not present in panels 312, such that panels 312 are substantially radio-transparent, or provide little or no radiation shielding compared to first and second shielding portions 302, 204.

Radiation shielding device 300 is adjustable from the first and second configurations by folding along one or more foldable regions 307, 309. For example, to place radiation shielding device 300 in the second, expanded configuration for use during a medical operation, a healthcare practitioner folds first shielding portion 302 about foldable region 307 and moves first shielding portion 302 towards surface 342 of second shielding portion 304. Next, the healthcare practitioner folds panels 312 inwardly along foldable region 309 (e.g., such that edges 340 align with edges 344 of second shielding portion 304). In some embodiments, the healthcare practitioner may then align and fasteners 308 with fasteners 310, and attach respective fasteners to one another.

In an example embodiment, radiation shielding device 300 is adjustable between collapsed and expanded configurations, and maintained in the expanded configuration, without bending or manipulating a malleable component or frame. In some example embodiments, radiation shielding device 300 does not include a malleable, shape-stable component or frame that is bent at the hinge point by a user when radiation shielding device 300 is adjusted between collapsed and expanded configurations.

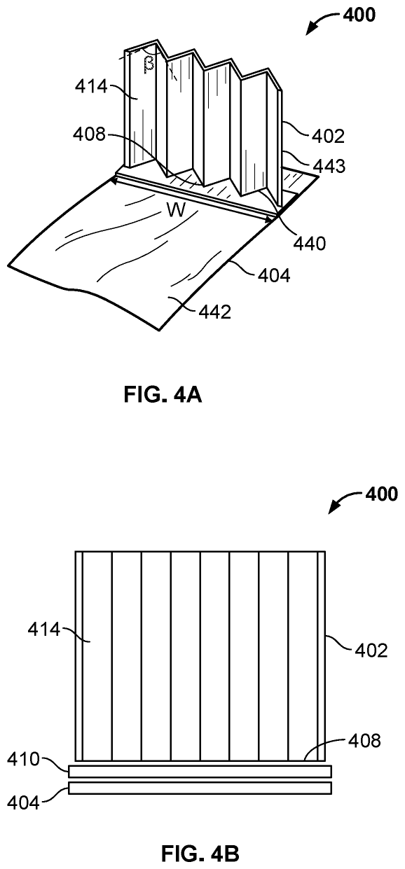

Referring now to FIGS. 4A and 4B, an example radiation shielding device 400 is shown. In an example embodiment, radiation shielding device 400 includes a first shielding portion 402 having foldable panels 414 (e.g., arranged in a zig-zag shape) and a second shielding portion 404. In various example embodiments, radiation shielding device 400 may include one or more features as described herein with reference to radiation shielding devices 100, 300 described above with reference to FIGS. 1-3B.

Radiation shielding device 400 is adjustable between a first configuration and a second configuration, such as a collapsed configuration and an expanded configuration (FIGS. 4A and 4B). In the first, collapsed configuration, first and second shielding portion may lay flat on one another. The first shielding portion 402 can be flattened in a substantially planar configuration (either in a folded configuration or an unfolded configuration or a combination of folded and unfolded configuration) such that major surfaces of the first and second shielding portions 402, 404 are parallel to one another.

In the expanded configuration, first shielding portion 402 may be folded along a plurality of foldable regions to impart a zig-zag shape. For example, foldable panels 414 define an angle beta (β) with respect to one another. In some embodiments, angle (β) may be between about 45° and 135°, 60° and 120°, or about 90°. In the first configuration, angle (B) may be about 90° such that the width of first shielding portion 402 spans about width w or less of second shielding portion 404. Such angles can provide a high degree of stability for first shielding portion 402 when radiation shielding device 400 is supported on a patient during a medical operation.

Radiation shielding device 400 include one or more attachment members 408 configured to maintain first shielding portion 402 in a selected position relative to second shielding portion 404. In various example embodiments, attachment member 408 includes one or more of an adhesive, hook-and-loop, snap-fit, button, toggle, ties, tongue-and-groove, keyed opening, complementary mechanical interface, ball-and-socket, quick-disconnect couplings, etc. In an example embodiment, attachment member 408 includes an adhesive strip covered by a release liner. At a time of use, the healthcare practitioner can remove the release liner and bring the adhesive strip into contact with complementary surface of second shielding portion 404 (e.g., an adhesive landing area) to attach first shielding portion 402 in a fixed location relative to second shielding portion 404. Alternatively or additionally, radiation shielding device 400 include a slot or groove having configured to receive second radiation shielding portion 402. For example, radiation shielding device include an attachment member 410 that includes a groove in a zig-zag pattern. The bottom edge 440 of first shielding portion 402 is engaged with the groove 410 to maintain first shielding portion 402 in a fixed position relative to second shielding portion 404.

In some optional embodiments, radiation shielding device 400 does not include an attachment member 410, and the first shielding portion 402 is attached to the second shielding portion 404 via attachment member 408 (e.g., directly attached).

Radiation shielding device 400 is constructed of one or more radio-opaque materials, including one or more materials described above with reference to FIGS. 1-2B, to provide radiation shielding for a healthcare practitioner. In an example embodiment, first and second shielding portions 402, 404 include radiation shielding materials (e.g., each have the same radiation-shielding material construction). In some example embodiments, the second shielding portion 404 includes areas of differing shielding levels. For example, area 442 on a first side of first shielding portion 402 (e.g., positioned away from a target area of a radiation source) includes radiation shielding materials to shield the healthcare practitioner, and an area 443 on a second side of first shielding portion 404 (e.g., positioned towards a target area of a radiation source) is radio-transparent or provides a reduced level of radiation shielding compared to area 442.

Radiation shielding device 400 is adjustable from the first configuration to the second configuration by connecting first and second shielding portions via attachment members 408, 410. In an example embodiment, first shielding portion is in a planar configuration when radiation shielding device 400 is in the first, collapsed configuration. The healthcare practitioner adjusts first shielding portion 402 into a zig-zag shape, such as by making multiple folds along the width of first shielding portion 402. The first shielding portion 402 is than attached to the second shielding portion 404 using attachment member 408 and/or 410 to maintain the first shielding portion 402 in a fixed positioned relative to second shielding portion 404. The folded, zig-zag shape of the first shielding portion 402, alone or together with attachment members 408, 410, facilitate a stable arrangement in which first and second shielding portions provide a zone of radiation shielding for a healthcare practitioner during a medical operation.

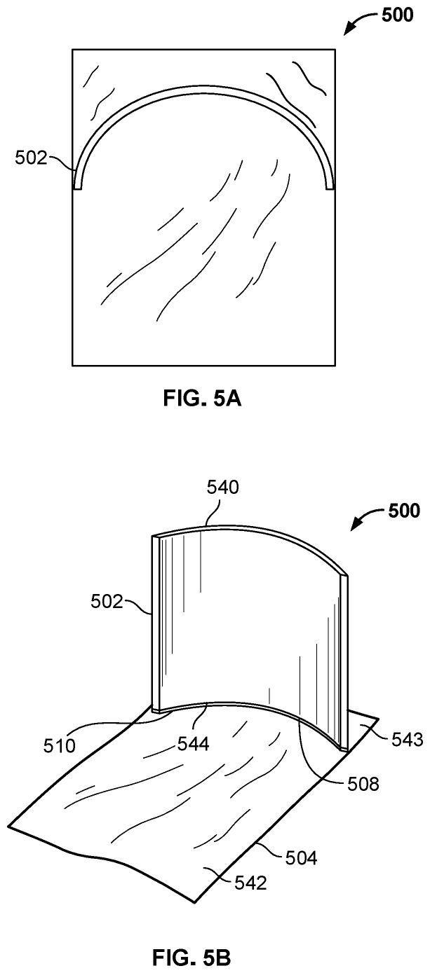

Referring now to FIGS. 5A and 5B, an example radiation shielding device 500 is shown. In an example embodiment, radiation shielding device 500 includes a first shielding portion 502 having a curved, top edge 540 (when in use), and a second shielding portion 504. In various example embodiments, radiation shielding device 500 may include one or more features as described herein with reference to radiation shielding devices 100, 300, 400 described above with reference to FIGS. 1-4B.

Radiation shielding device 500 is adjustable between a first configuration and a second configuration, such as a collapsed configuration and an expanded configuration (FIGS. 5A and 5B). In the first, collapsed configuration, first and second shielding portions 502, 504 may be unconnected (or only partially connected along bottom edge 544) and in a substantially flat configuration relative to one another. For example, the first shielding portion 502 can be flattened in a substantially planar configuration such that major surfaces of the first and second shielding portions 502, 504 are parallel to one another. In some embodiments, first shielding portion 502 is substantially rigid and has a predetermined or defin curvature (e.g., along top edge 540). In some embodiments, curved, top edge 540 facilitates alignment with a radiation source having a complementary shape, and/or provides a predictable zone of radiation shielding for the healthcare practitioner.

The first configuration facilitates compact packaging for efficient transport and storage. Multiple first shielding portions 502 can be individual wrapped and sterile packaging and stacked, and multiple second shielding portions 504 can be individual wrapped in sterile packaging and stacked, such that multiple first and second shielding portions can be transported and stored in a relatively compact volume.

In the expanded configuration, first shielding portion 502 has a curved configuration. In some embodiments, first shielding portion 502 is conformable and shape-stable such that first shielding portion 502 can be bent and maintained in a selected shape (e.g., a selected curvature) at the time of use. A healthcare practitioner can manipulate first shielding portion 502 from a flat configuration to a curved configuration (FIGS. 5A and 5B), the second shielding portion 502 maintains the curved configuration. The curved configuration of first shielding portion 502 can provide a high degree of stability when radiation shielding device 400 is supported on a patient during a medical operation.

Radiation shielding device 500 preferably includes one or more attachment members 508 configured to maintain first shielding portion 502 in a selected position relative to second shielding portion 504. In various example embodiments, attachment member 508 includes one or more of an adhesive, hook-and-loop, snap-fit, button, toggle, ties, tongue-and-groove, keyed opening, complementary mechanical interface, ball-and-socket, quick-disconnect couplings, etc. In an example embodiment, attachment member 508 includes an adhesive strip covered by a release liner. At a time of use, the healthcare practitioner can remove the release liner and bring the adhesive strip into contact with complementary surface of second shielding portion 504 (e.g., an adhesive landing area) to attach first shielding portion 502 in a fixed location relative to second shielding portion 504. Alternatively or additionally, radiation shielding device 500 include a slot or groove having configured to receive second radiation shielding portion 502. For example, second radiation shielding 504 includes a groove in a curved shape complementary to the curvature of first shielding portion 502. The bottom edge 544 of first shielding portion 502 is engaged with the groove to maintain first shielding portion 502 in a fixed position relative to second shielding portion 504.

Radiation shielding device 500 is constructed of one or more radio-opaque materials, including one or more materials described above with reference to FIGS. 1-2B, to provide radiation shielding for a healthcare practitioner. In an example embodiment, first and second shielding portions 502, 504 include radiation shielding materials (e.g., each have the same radiation-shielding material construction). In some example embodiments, the second shielding portion 504 includes areas of differing shielding levels. For example, area 542 on a first side of first shielding portion 502 (e.g., positioned away from a target area of a radiation source) includes radiation shielding materials to shield the healthcare practitioner, and an area 543 on a second side of first shielding portion 504 (e.g., positioned towards a target area of a radiation source) is radio-transparent or provides a reduced level of radiation shielding compared to area 542.

Radiation shielding device 500 is adjustable from the first configuration to the second configuration by connecting first and second shielding portions along the bottom edge 544 via attachment members 508. In an example embodiment, first shielding portion is in a planar configuration when radiation shielding device 500 is in the first, collapsed configuration. The healthcare practitioner adjusts first shielding portion 502 into a curved shape. The first shielding portion 502 is than attached to the second shielding portion 504 using attachment member 508 to maintain the first shielding portion 502 in a fixed positioned relative to second shielding portion 504. The curved shape of the first shielding portion 502, alone or together with attachment member 408, facilitate a stable arrangement in which first and second shielding portions provide a zone of radiation shielding for a healthcare practitioner during a medical operation.

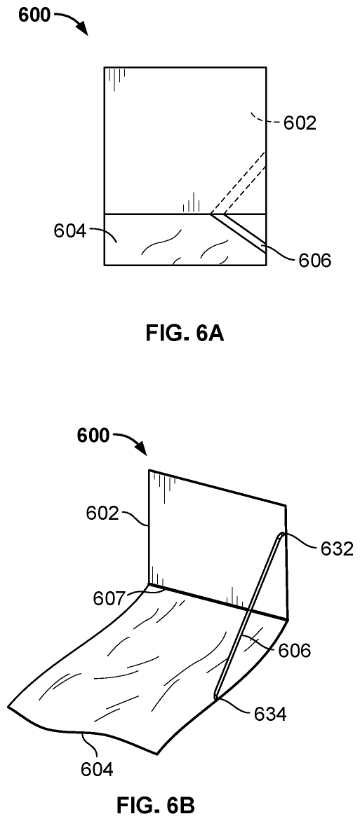

Referring now to FIGS. 6A and 6B, an example radiation shielding device 600 is shown. In an example embodiment, radiation shielding device 600 includes a first shielding portion 602, a second shielding portion 604, and one or more attachment members 606 (a second attachment member is included, e.g., on the other side of the device, in some embodiments). In various example embodiments, radiation shielding device 600 may include one or more features as described herein with reference to radiation shielding devices 100, 300, 400, 500 described above with reference to FIGS. 1-5B.

Radiation shielding device 600 is adjustable between a first configuration and a second configuration, such as a collapsed configuration (FIG. 6A) and an expanded configuration (FIG. 6B). In an example embodiment, when in the first, collapsed configuration, radiation shielding device 600 is foldable (e.g., about a folding axis defined through foldable region 607) such that the first shielding portion 602 at least partially overlaps with second shielding portion 604, and/or major surfaces of the first and second shielding portions 602, 604 are parallel to one another. Alternatively or additionally, first shielding portion 602 can be foldable to extend away from second shielding portion 604. For example, first and second shielding portions 602, 604, and/or panels 612, are co-planar with one another in such an arrangement.

In an example embodiment, foldable region 607 includes a hinge or biased component that biases radiation shielding device 600 in a particular configuration. For example, foldable region 607 biases first shielding portion 602 toward second shielding portion 604. Foldable region 607 may be biased via a spring hinge, leaf hinge, biased stiffener, etc.

One or more attachment member 606 maintain the radiation shielding device 600 in an expanded configuration. For example, attachment member 606 maintains the radiation shielding device in a selected configuration. In an example embodiment, attachment member 606 comprises a flexible strap. Tension of strap 606 counteracts force from the biased foldable region 607, maintaining first shielding portion in the expanded configuration. For example, tension of strap 606 limits relative movement of first shielding portion 602 away from relative to second shielding portion 604 beyond a predetermined point. In various example embodiments, attachment member 606 limits angle (α) between first and second shielding portions to less than 135° less than 110°, less than 100°, or about 90°. Alternatively or additionally, attachment member 606 can include a rod, such as an adjustable or telescopic rod (e.g., a rod having an adjustable length), or a rigid, non-adjustable rod (e.g., that provides support in both tension and compression). Alternatively or additionally, attachment member 606 can include a triangular piece of fabric or other foldable material.

First shielding portion 602 may be positioned substantially vertically with respect to the second shielding portion 604, when in the second, expanded configuration. An angle between first and second shielding portions 602, 604, can be adjusted by a healthcare practitioner by adjusting the length of an attachment member 606, or locations where attachment member 606 attaches with first and second shielding portions.

Attachment member 606 is connected to first shielding portion 602 at attachment locations 632 and to second shielding portion 604 at attachment locations 634. In some embodiments, attachment locations 632 and 634 comprise one or more of adhesive, hook-and-loop, snap-fit, button, toggle, ties, keyed opening, complementary mechanical interface, ball-and-socket, quick-disconnect couplings, etc. When in the collapsed configuration, attachment member 606 may be folded inwards such that attachment members are sandwiched between first and second shielding portions 602, 604. Alternatively or additionally, attachment member 606 may be folded outwards (e.g., not positioned between the first shielding portion 602 and the second shielding portion 604). In some embodiments, attachment member 606 is removably connectable at one or more of attachment locations 632, 634. For example, radiation shielding device 600 may be packaged in the first configuration (e.g., collapsed configuration) in which attachment member 606 is disconnected. A healthcare practitioner can adjust the radiation shielding device 600 into the second, expanded configuration by connecting attachment member 606 to attachment locations 632, 634, for example.

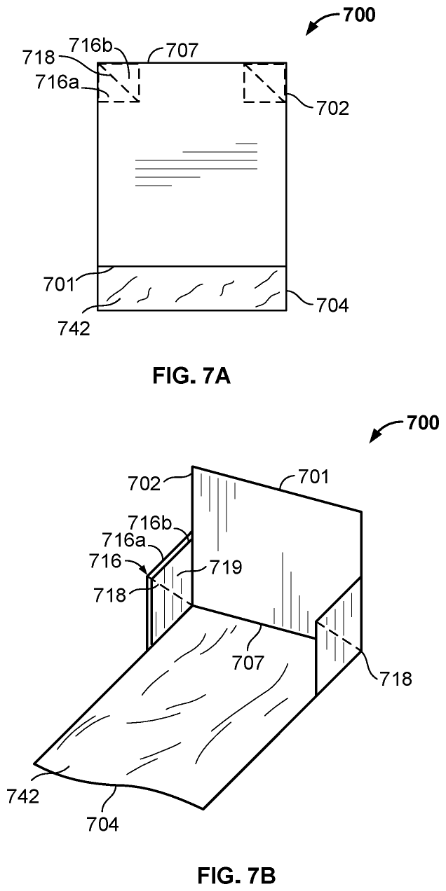

Referring now to FIGS. 7A and 7B, an example radiation shielding device 700 is shown. In an example embodiment, radiation shielding device 700 includes a first shielding portion 702, a second shielding portion 704, and side panels 716. In various example embodiments, radiation shielding device 700 may include one or more features as described herein with reference to radiation shielding devices 100, 300, 400, 500, 600 described above with reference to FIGS. 1-6.

Radiation shielding device 700 is adjustable is adjustable between a first configuration and a second configuration, such as a collapsed configuration (FIG. 7A) and an expanded configuration (FIG. 7B). In an example embodiment, when in the first, collapsed configuration, first shielding portion 702 at least partially overlaps with second shielding portion 304, and/or panels 716. Alternatively, first shielding portion 302 can extend away from second shielding portion 704 such that first and second shielding portions 702, 704, and/or panels 712, are co-planar with one another.

Radiation shielding device 700 includes side panels 716 that are foldable to facilitate the first, collapsed configuration of radiation shielding device 700, and expandable to extend between first and second shielding portions 702, 704, to maintain first shielding portion in a position relative to second shielding portion when radiation shielding device is in the second, expanded configuration. Side panels 716 include a folding axis 718. In the collapsed configuration, side panels 716 are folded along folding axis 718 (e.g., such that respective halves on opposite sides of fold axis 718 overlap and collapse on one another. In the expanded configuration, side panels 716 extend between first and second shielding portions 702, 704, and provide structural support that resists relative movement between first and second shielding portions 702, 704.

In some embodiments, side panels 716 have a two-layer construction. For example, side panels 716 includes a first side panel 716a that includes folding axis 718, and a second side panel 716b that does not include a folding axis. The first side panel 716a is attached to second side panel 716a on a first side 719 of folding axis 718. In some embodiments, first side panel 716a is directly attached to only one of first shielding portion 702 and second shielding portion 704, and second side panel is directly attached to only one of the other of first shielding portion 702 and second shielding portion 704 (e.g., such that both first and second side panels 716a, 716b are not each directly attached to both first and second shielding portions 702, 704). In a collapsed configuration, first side panel 716a is folded about folding axis 718 and second side panel 718 is not folded. In the expanded configuration (FIG. 7B), the second side panel 716b promotes structural stability and inhibits bending of first side panel 716a.

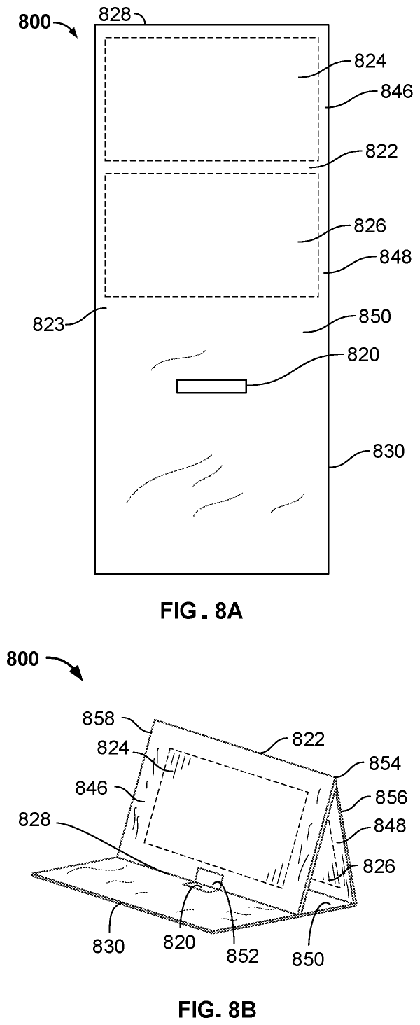

Referring now to FIGS. 8A and 8B, an example radiation shielding device 800 is shown. In various example embodiments, radiation shielding device 800 may include one or more features as described herein with reference to radiation shielding devices 100, 300, 400, 500, 600, 700 described above with reference to FIGS. 1-7.

In an example embodiment, radiation shielding device 800 is adjustable between a first configuration, such as a collapsed configuration (FIG. 8A) and an expanded configuration (FIG. 8B). Radiation shielding device 800 includes two or more foldable regions 822, 823 separating first, second, and third sections 846, 848, and 850. In an example embodiment, when in the first, collapsed configuration, radiation shielding device 800 is in a substantial flat, planar configuration. For example, first, second, and third sections 846, 848, and 850. Alternatively or additionally, when in the first configuration, radiation shielding device 800 can be folded along foldable regions 822, 823 such that first, second, and/or third sections 846, 848, and 850 at least partially overlap.

Referring now to FIG. 8B, radiation shielding device 800 is shown in a second configuration. In the second configuration, radiation shielding device 800 is folded into a three-dimensional shape that at least partially supports radiation shielding device 800 in the second configuration. For example, radiation shielding device 800 is folded about foldable region 823 such that second section 848 is oriented at an angle relative to third section 850, and folded about foldable region 822 such that first section 846 is oriented at an angle relative to second section 848. At least portions of the first, second, and third sections form a triangular shape. The folded shape of the radiation shielding device 800 imparts structure that at least partially supports second section 848 in the orientation relative to third section 850. The second section 848 is oriented at an angle such that the second section extends generally upwardly from the third section 850. The second section 848 extends at least partially between the patient and a radiation source to provide radiation shielding in a substantially vertical direction, and the third section 850 is located at least partially over a patient to provide radiation shielding in a substantially horizontal direction.

In an example embodiment, radiation shielding device includes an attachment member 820 configured to interact with an edge 828 or other feature of first section 824 when in the second, expanded configuration. For example, attachment member 820 can include a slot, ridge, magnet, etc. configured to interact with edge 828. The weight of first and/or second sections 846, 848 can exert a force that pushes edge 828 towards attachment member 820, maintaining the edge 828 in engagement with attachment member 820.

Alternatively or additionally, an attachment member 852 is disposed at or near edge 828 such that when radiation shielding device 800 is in the second configuration (e.g., the operating configuration), attachment member 852 engages with attachment member 820. In various example embodiments, attachment members 820, 852 include one or more of an adhesive, hook-and-loop, snap-fit, button, toggle, ties, tongue-and-groove, keyed opening, complementary mechanical interface, ball-and-socket, quick-disconnect couplings, magnet (e.g., interlocking male and female magnetic connectors), clasp, etc.

To adjust radiation shielding device 800 from the first configuration to the second configuration, a healthcare practitioner folds radiation shielding device 800 about a folding axis defined through foldable region 822 and about a folding axis defined through foldable region 823, and moves first section 846 towards attachment member 820 until attachment member 852 is aligned with and engages attachment member 820. Third section 850 forms a base supportable by an object (e.g., a patient) and provides support to the first and second sections 846, 848. First and second sections 846, 848 form a triangular structure having a portion of foldable region 822 as its vertex 854 and defining a first face 856 that may face radiation emitted from a radiation source and a second face 858 that may face a healthcare practitioner during a medical operation.

In an example embodiment, radiation shielding device 800 includes one or more stiffening layers that impart structure to some or all of radiation shielding device 800. For example, first and second sections 846, 848 include one or more stiffening layers 824, 826. Stiffening layers 824, 826 can include a substantially rigid layer or component, such as a plastic, metal, or other stiffening layer. In some embodiments, stiffening layers 824, 826 are radio-opaque, and can be provided by a radiation shielding layer. In some embodiments, stiffening layers 824, 826 are not malleable (e.g., and return substantially to an original shape when bent).

In an example embodiment, stiffening layers 824, 826 are present throughout first and second sections 846, 848, respectively. For example, stiffening layers 824, 826 have dimensions the same or similar as outer dimensions of first and second sections 846, 848. In some embodiments, stiffening layers 824, 846 include one or more discrete sheets, rods, frame components, etc., that are spaced apart in first and second sections 846, 848. In an example embodiment, stiffening layers 824, 826 are internal components located between outer layers of radiation shielding device 800. Alternatively or additionally, stiffening layers 824, 826 may include stiffening components laminated to an exterior of first and second sections 846, 848.

Radiation shielding device 800 is constructed of one or more radio-opaque materials, including one or more materials described above with reference to FIGS. 1-2B, to provide radiation shielding for a healthcare practitioner. In an example embodiment, first, second, and third sections 846, 848, 850 include radiation shielding materials (e.g., each have the same radiation-shielding material construction). Alternatively, second and third sections 848, 850, include one or more radiation shielding materials that are not present in first section 846, such that first section 846 is substantially radio-transparent, or provides little or no radiation shielding compared to second and third sections 848, 850.

In an example embodiment, radiation shielding device 800 is adjustable between collapsed and expanded configurations, and maintained in the expanded configuration, without bending or manipulating a malleable component or frame. In some example embodiments, radiation shielding device 800 does not include a malleable, shape-stable component or frame that is bent by a user when radiation shielding device 800 is adjusted between collapsed and expanded configurations.

While this specification contains many specific implementation details, these should not be construed as limitations on the scope of any technology or of what may be claimed, but rather as descriptions of features that may be specific to particular embodiments. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Particular embodiments of the subject matter have been described. Other embodiments are within the scope of the following claims.

1.-40. (canceled)

41. A radiation shielding device, comprising:
a first radiation shielding portion;
a second radiation shielding portion attached to the first radiation shielding portion; and
an attachment member,
wherein the radiation shielding device is adjustable between a first configuration in which the first radiation shielding portion is in a first orientation relative to the second radiation shielding portion and a second configuration in which the first radiation shielding portion is in a second orientation relative to the second radiation shielding portion, and
wherein the attachment member is configured to maintain the first radiation shielding portion in the second orientation relative to the second radiation shielding portion.

42. The radiation shielding device of claim 41, wherein first configuration is a planar or collapsed configuration and the second configuration is an expanded configuration in which the first radiation shielding portion is disposed at an angle relative to the first shielding radiation portion, and wherein the angle is between 75° and 105°.

43. The radiation shielding device of claim 42, wherein the attachment member comprises a first panel attached to the first radiation shielding portion, and wherein the first panel is attachable to the second radiation shielding portion.

44. The radiation shielding device of claim 43, wherein the first panel is removably attachable to the second radiation shielding portion by a fastener selected from the group consisting of an adhesive, hook-and-loop, snap-fit, button, toggle, ties, and tongue-and-groove fastener.

45. The radiation shielding device of claim 43, wherein the first panel is foldable relative to the first radiation shielding portion such that a major surface of the first panel is parallel to a major surface of the first radiation shielding portion when the radiation shielding device is in the collapsed configuration, and angled relative to the first radiation shielding portion when the radiation shielding device is in an expanded configuration.

46. The radiation shielding device of claim 45, wherein the major surface of the first panel is parallel with a major surface of the second radiation shielding portion when the radiation shielding device is in the collapsed configuration, and perpendicular to the major surface of the second radiation shielding portion when the radiation shielding device is in the expanded configuration.

47. The radiation shielding device of claim 41, wherein the first and second radiation shielding portions comprise a radiation shielding drape material, and wherein the attachment member comprises a flexible strap that is connected to the first radiation shielding portion proximate a first end of the flexible strap and connected to the second radiation shielding portion proximate a second end of the flexible strap.

48. The radiation shielding device of claim 41, wherein the attachment member comprises a fastener configured to attach a bottom surface of the first radiation shielding portion to the second radiation shielding portion, and wherein the fastener is selected from the group consisting of adhesive, hook-and-loop, snap-fit, button, toggle, ties, and tongue-and-groove fastener.

49. The radiation shielding device of claim 41, comprising a plurality of foldable regions, wherein the first radiation shielding portion comprises a surface having a definable fixed curvature, wherein the first radiation shielding portion comprises a plurality of surfaces in a zig-zag pattern, and wherein the first radiation shielding portion is a separate component from the second radiation shielding portion.

50. The radiation shielding device of claim 41, wherein the radiation shield includes a passage defined through a thickness of the first radiation shielding portion, the passage configured to allow medical tool to pass from a first side of the first radiation shielding portion to a second side of the first radiation shielding portion.

51. The radiation shielding device of claim 41, wherein the first radiation shielding portion is self-supporting when arranged in a vertical orientation, and wherein the second radiation shielding portion is non-shape stable.

52. The radiation shielding device of claim 51, wherein the first and second radiation shielding portions are integrally formed as a unitary component and comprise an outer fabric layer, an internal radiation shielding layer, and a stiffening layer.

53. The radiation shielding device of claim 41, wherein the first configuration is a collapsed configuration in which the radiation shielding device is flat, and the second configuration is an expanded configuration in which the radiation shielding device forms a triangle shape, and wherein vertices of the triangle shape are defined by a first foldable region, a second foldable region, and a perimeter edge of the radiation shielding device.

54. A radiation shielding device, comprising:
a radiation shielding drape comprising a first section, a second section, and a third section, the first section comprising a first stiffening component and the second section comprising a second stiffening component;
a first foldable region between the first and second sections; and
a second foldable region between the second and third sections,
wherein the radiation shielding drape is foldable along the first and second foldable regions to adjust the radiation shielding device between a first configuration and a second configuration.

55. The radiation shielding device of claim 54, wherein the radiation shielding drape forms a triangle shape in the second configuration, and wherein the first and second sections are rigid, and wherein the third section is flexible.

56. The radiation shielding device of claim 55, comprising an attachment member configured to attach the first section to the third section, wherein the attachment member is configured to attach an edge of the first section to the third section, and wherein the attachment member is a magnet, a tab, or a fastener.

57. The radiation shielding device of claim 54, wherein the third section is supportable on an object while the first and second sections extend upwardly above the third section, and wherein the object is a patient.

58. The radiation shielding device of claim 57, comprising a passage defined through a thickness of the radiation shielding drape, the passage configured to allow a medical tool to pass from a first side of the radiation shielding drape to a second side of the radiation shielding drape.

59. A method of shielding radiation, comprising:
adjusting a first radiation shielding portion of a radiation shielding device relative to a second radiation shielding portion of the radiation shielding device;
positioning an attachment member to extend between the first radiation shielding portion and the second radiation shielding portion; and
positioning the radiation shielding device on a patient,
wherein positioning the attachment member comprises folding a panel of the radiation shielding device to at least partially maintain the first radiation shielding portion in a selected position relative to the second radiation shielding portion.

60. A radiation shielding device, comprising:
first and second radiation shielding portions; and
means for maintaining the first radiation shielding portion in a selected position relative to the second radiation shielding portion,
wherein the radiation shielding device does not include a malleable, shape-stable component at a hinge region between the first and second radiation shielding portions.
